(19) United States
(12) Patent Application Publication
(10) Pub. No.: US 2024/0028176 A1
(43) Pub. Date: Jan. 25, 2024

(54) SYSTEMS, METHODS, AND GRAPHICAL USER INTERFACES FOR DIGITALLY RECREATING DISPLAY STATES OF DIAGNOSTIC TEST RESULTS

(71) Applicant: EMED LABS, LLC, Miami, FL (US)

(72) Inventors: Zachary Carl Nienstedt, Wilton Manors, FL (US); Igor Javier Rodriguez, Miami, FL (US)

(21) Appl. No.: 18/338,252

(22) Filed: Jun. 20, 2023

Related U.S. Application Data
(60) Provisional application No. 63/367,970, filed on Jul. 8, 2022.
(60) Provisional application No. 63/366,554, filed on Jun. 17, 2022.

(51) Int. Cl. G06F 3/04812; G06F 3/0482

(52) U.S. Cl. CPC G06F 3/04812; G06F 3/0482

(57) ABSTRACT

Systems, methods, and graphical user interfaces for recreating the display states of diagnostic test results are disclosed herein. In some embodiments, a computer-implemented method is disclosed for displaying a graphical user interface with a plurality of non-overlapping bands that each have a display state selected from among a set of possible display states. The display state of each band may be initially set to a default state selected from among the set of possible display states. The display state of each band may be quickly changed by a user input, allowing for the quick recreation of the overall display state of a diagnostic test result. The display states of the bands can be converted into a machine-readable format for more-efficient communication and storage.

### CROSS-REFERENCE TO RELATED APPLICATIONS

This application claims the benefit of U.S. Provisional Patent Application No. 63/366,554, entitled “METHODS, SYSTEMS, AND DEVICES FOR RECREATING RESULTS FOR DIAGNOSTIC TESTS,” filed Jun. 17, 2022, the contents of which are incorporated by reference herein in their entirety. This application also claims the benefit of U.S. Provisional Patent Application No. 63/367,970, entitled “METHODS, SYSTEMS, AND DEVICES FOR RECREATING RESULTS FOR DIAGNOSTIC TESTS,” filed Jul. 8, 2022, the contents of which are incorporated by reference herein in their entirety.

### TECHNICAL FIELD

The embodiments of the disclosure generally relate to systems, methods, and graphical user interfaces for recreating the display states of diagnostic test results, such as the display states of the results window and the result indicators. In the context of self-administered diagnostic testing, the systems, methods, and graphical user interfaces disclosed herein may enable a user to quickly recreate the display states of a diagnostic test result for submission, interpretation, and/or verification.

### BACKGROUND

Remote or at-home healthcare testing and diagnostics can solve or alleviate some problems associated with in-person testing. For example, health insurance may not be required, travel to a testing site is avoided, and tests can be completed at a testing user's convenience. However, remote or at-home testing introduces various additional logistical and technical issues, such as relying on a user's interpretation of test results. The use of telehealth technologies can alleviate some of these issues by allowing for long distance patient and health provider contact, such as via a user or patient's personal user device (e.g., a smartphone, tablet laptop, personal computer, or other device). For example, a user or patient can interact with a remotely located medical care provider using live video, audio, or text-based chat through the personal user device in order to receive guidance and/or oversight of the testing procedures remotely.

However, it may not always be feasible to provide real-time, synchronous monitoring and guidance via live video or audio. For instance, those formats may make it overwhelming, time-consuming, and inefficient to monitor a large quantity of remote or at-home diagnostic testing being performed simultaneously. Also, such communication occurs over a network (e.g., a cellular or internet network), which can have poor signal quality. Alternatively, text-based chat may be poorly suited for accurately communicating and reviewing the results of remote or at-home diagnostic testing, especially when a visual component is involved.

This can be especially problematic in some instances, such as when the results of diagnostic tests need to be determined but their interpretation is up to the user (e.g., cannot be interpreted by an online proctor or computer vision algorithm), and the user is normally a naive actor who is not trained in results interpretation. Furthermore, it can be problematic to extract and store the results of the diagnostic tests, since the aforementioned approaches may involve large data transmissions and/or large data records that contain a mountain of extraneous data.

Thus, there exists a need to enable users of remote or at-home diagnostic tests to easily recreate and communicate the display states of their diagnostic test results for review, comparison, and storage.

### SUMMARY

For purposes of this summary, certain aspects, advantages, and novel features are described herein. It is to be understood that not necessarily all such advantages may be achieved in accordance with any particular embodiment. Thus, for example, those skilled in the art will recognize the disclosures herein may be embodied or carried out in a manner that achieves one or more advantages taught herein without necessarily achieving other advantages as may be taught or suggested herein.

All of the embodiments described herein are intended to be within the scope of the present disclosure. These and other embodiments will be readily apparent to those skilled in the art from the following detailed description, having reference to the attached figures. The invention is not intended to be limited to any particular disclosed embodiment or embodiments.

The embodiments of the disclosure generally relate to systems, methods, and graphical user interfaces for recreating the display states of diagnostic test results, such as the display states of the results window and the result indicators associated with a lateral flow test.

For example, in some embodiments, a computer-implemented method is disclosed for digitally recreating a display state of a test result in a graphical user interface. The method may involve displaying a graphical user interface on a screen (e.g., on a user device), and the graphical user interface may comprise a plurality of non-overlapping bands (e.g., lines) that each have a display state selected from among a set of possible display states. These display states may differ on various attributes/dimensions, such as color, color intensity, and so forth. The display state of each band of the plurality of bands may be initially set to a default state selected from among the set of possible display states. The overall appearance of the graphical user interface may be configured to resemble a real-life diagnostic test, such as a results window of a lateral flow test.

A user input associated with a particular band can be detected, and the displayed graphical user interface may be updated by changing the display state of that band to a different state selected from among the set of possible display states. The user can continue providing user inputs in this manner until the display states of all the bands in the graphical user interface are set to resemble the overall display state of their test result. The user can then provide additional user input to indicate that the display states of all the bands in the graphical user interface sufficiently resemble and recreate the display state of the test result.

The display states of the bands in the graphical user interface can be converted into a machine-readable format for communication and storage. The converted display states can be used to generate a possible interpretation of the test result that can be used to provide suggestions or recommendations to the user. An image or snapshot of the test result can also be captured (e.g., via a camera). The image of the test result and the converted display states can be transmitted for remote comparison, verification, and storage. In some cases, a similar graphical user interface can be used to enable a supervising user to compare the image of the test result to the converted display states from the user submission, or even to recreate the display state of the test result on their own device.

### BRIEF DESCRIPTION OF THE DRAWINGS

These and other features, aspects, and advantages of the disclosure are described with reference to drawings of certain embodiments, which are intended to illustrate, but not to limit, the present disclosure. It is to be understood that the accompanying drawings, which are incorporated in and constitute a part of this specification, are for the purpose of illustrating concepts disclosed herein and may not be to scale.

FIG. 1 depicts a non-limiting example of the results re-creation widget mimicking the results of a real diagnostic test.

FIG. 2 is an example schematic diagram of a user inputting their diagnostic test results on the patient-facing side of the results re-creation widget.

FIGS. 3A-B depicts a sample user interface of the patient-facing side of the results re-creation widget.

FIG. 4 shows the patient-facing side of the results re-creation widget displaying results from the real diagnostic test in both graphical and tabular form.

FIG. 5 depicts a sample perspective view of the patient-facing side of the results re-creation widget in a mobile application.

FIGS. 6A-6B depict sample user interfaces of the proctor-facing side of the results re-creation widget mimicking the results of a real diagnostic test.

FIG. 7 presents a block diagram illustrating an embodiment of a computer hardware system configured to run software for implementing one or more embodiments of the health testing and diagnostic systems, methods, and devices disclosed herein.

### DETAILED DESCRIPTION

Although several embodiments, examples, and illustrations are disclosed below, it will be understood by those of ordinary skill in the art that the inventions described herein extend beyond the specifically disclosed embodiments, examples, and illustrations and includes other uses of the inventions and obvious modifications and equivalents thereof. Embodiments of the inventions are described with reference to the accompanying figures, wherein like numerals refer to like elements throughout. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

In some instances, a problem arises when results of diagnostic tests need to be determined, but their interpretation is up to the user (e.g., cannot be interpreted by an online proctor or computer vision algorithm). The user is normally a naive actor who is not trained in results interpretation. Therefore, complicated logic trees must be created to arrive at the correct result. For example, a lateral flow test with 2 possible lines has 4 states. However, a test with 4 possible lines has 16 states.

This disclosure presents an intuitive way to shortcut these logic trees, enabling users and proctors of at home diagnostic tests to easily recreate and communicate the state of a diagnostic test result display in a digital manner for easy communication, comparison, and storage. In some embodiments, the methods and systems can be customizable to almost any diagnostic test imaginable. It can also be a tool to compare user and proctor interpretations of a test and gather data for training machine learning models.

It has been noted that design of computer user interfaces “that are useable and easily learned by humans is a non-trivial problem for software developers.” (Dillon, A. (2003) User Interface Design. MacMillan Encyclopedia of Cognitive Science, Vol. 4, London: MacMillan, 453-458.) The present disclosure describes various embodiments of interactive and dynamic user interfaces that are the result of significant development. This non-trivial development has resulted in the user interfaces described herein which may provide significant cognitive and ergonomic efficiencies and advantages over previous systems. The interactive and dynamic user interfaces include improved human-computer interactions that may provide reduced mental workloads, improved decision-making, reduced work stress, and/or the like, for a user. For example, user interaction with the interactive user interfaces described herein may enable both users and computers to more quickly and accurately access, navigate, enrich, assess, and digest large numbers of data items than previous systems.

Further, the interactive and dynamic user interfaces described herein are enabled by innovations in efficient interactions between the user interfaces and underlying systems and components. For example, disclosed herein are improved methods of displaying bands that correspond to the lines on a results window of an actual diagnostic test, changing the display states of the bands based on user interactions until the bands resemble the lines of the actual diagnostic test (e.g., in a quick and streamlined manner with a limited number of display states that need to be considered, so as to reduce excessive user interactions and user decision making), and processing or converting the display states into machine-readable formats for improved efficiency (e.g., reduced data storage, faster communication, faster processing). The interactions and presentation of data via the interactive user interfaces described herein may accordingly provide cognitive and ergonomic efficiencies and advantages over previous systems.

Various embodiments of the present disclosure provide improvements to various technologies and technological fields. For example, existing interfaces for recreating the appearance of a real-life object are limited in various ways, and various embodiments of the disclosure provide significant improvements over such technology. Additionally, various embodiments of the present disclosure are inextricably tied to computer technology. In particular, various embodiments rely on detection of user inputs via graphical user interfaces, alteration and manipulation of display states of user interface elements in the graphical user interfaces based on the detected user inputs, generation and transmission of image files by capturing camera snapshots and screen grabs, generation of data items in tables or varying machine-readable formats by processing and converting the display states, and improvement of computer vision applications and machine learning models based on the data items, and/or the like. Such features and others are intimately tied to, and enabled by, computer technology, and would not exist except for computer technology. For example, the interactions with displayed data described below in reference to various embodiments cannot reasonably be performed by humans alone, without the computer technology upon which they are implemented. Further, the implementation of the various embodiments of the present disclosure via computer technology enables many of the advantages described herein, including more efficient interaction with, and presentation of, various types of data contained or associated with diagnostic test results.

In some embodiments, the graphical user interfaces disclosed herein may be used to digitally recreate the display state of any suitable diagnostic test result. In some embodiments, the graphical user interface may be referred to as a results re-creation widget or the graphical user interface may include a results re-creation widget, which may be an interactable digital object that can be designed to resemble the results window of the real diagnostic test. The graphical user interfaces disclosed herein may be especially useful for use with diagnostic tests that communicate results that require additional interpretation, such as through lines or other unclear indications.

In some embodiments, the graphical user interfaces disclosed herein may be used to digitally recreate the display state of any lateral flow test result (e.g., the results window). A lateral flow test is an assay also known as a lateral flow device (LFD), lateral flow immunochromatographic assay, or rapid test. It is a simple device intended to detect the presence or absence of a target analyte in a liquid sample, and it is widely used in medical diagnostics in the home, at the point of care, and in the laboratory. For example, it can be used to detect specific target molecules, such as molecules associated with pathogens and diseases, gene expression or biomarkers in humans (e.g., hormones and other proteins), chemicals or toxins, and so forth. It can test a variety of samples, like urine, blood, saliva, sweat, serum, and other fluids. Lateral flow tests can also be used in other contexts beside medical diagnostics, such as food safety and environmental monitoring.

Accordingly, the graphical user interfaces disclosed herein could be used for diagnostic tests in any of these cases and contexts; some non-limiting examples of specific diagnostic tests that the graphical user interfaces disclosed herein could be used with include COVID-19 diagnostic tests or drug diagnostic tests for detecting the presence of a drug (e.g., illegal or prescription drugs) in a sample. For example, disclosed herein are improved methods of displaying bands that correspond to the lines on a results window of an actual diagnostic test, changing the display states of the bands based on user interactions until the bands resemble the lines of the actual diagnostic test (e.g., in a quick and streamlined manner with a limited number of display states that need to be considered, so as to reduce excessive user interactions and user decision making), and processing or converting the display states into machine-readable formats for improved efficiency (e.g., reduced data storage, faster communication, faster processing). The interactions and presentation of data via the interactive user interfaces described herein may accordingly provide cognitive and ergonomic efficiencies and advantages over previous systems.

In some embodiments, the graphical user interface may have non-overlapping lines (also referred to as bands) that correspond to lines in the results window of a lateral flow test. For example, a particular lateral flow test may have a results window with one or more test lines and a control line that can change color to visually display a positive or negative result, and the lines may be of varying color intensity (e.g., strong, weak, no color). Accordingly, the lines in the graphical user interface may correspond to the lines of that lateral flow test, and they may be displayable in different display states that are within the range of possibilities that can visually occur for the lines of the lateral flow test. The display states of the lines in the graphical user interface may differ on one or more dimensions, such as color, color intensity, color saturation, shade, tint, hue, tone, pattern, texture, highlights, and so forth.

In some embodiments, the lines may be shown against a background with a color corresponding to a color of the membrane (e.g., white) in the results window of a lateral flow test. In some embodiments, the lines may have a display state in which the color of the line matches the color of the background (e.g., so that there is no visually detectable or distinguishable line), like how a line in a result window of a lateral flow test may not be detectable from the membrane (e.g., corresponding to an unused test or a negative result). In some embodiments, the lines may have a display state in which the line is slightly or barely distinguishable from the background (e.g., a color of the line is slightly different from the color of the background), enough so that a user of the graphical user interface can see that the line is an element of the graphical user interface (and thus be possibly motivated to interact with it). In some embodiments, the display state of a line may temporarily change as a user hovers over the line (e.g., with their mouse cursor). For instance, the line may temporarily change color, become highlighted, and so forth, in order to catch the user's attention and suggest that the line may be an interactable element of the graphical user interface.

In some embodiments, each line in the graphical user interface may have a display state that is selected from among a set of possible display states. In some embodiments, there can be a single set of possible display states that the display states for all the lines are selected from. For instance, there may be a total of three different configurations that the display state of any of the lines could possibly take. Alternatively, in other embodiments, there could be multiple sets of possible display states that the display states for all the lines are selected from. For example, the display state of each line may be selected from its own pool of possible display states. In some embodiments, some or all of the possible display states may fall within the range of possibilities that can visually occur for the lines of the corresponding lateral flow test that the graphical user interface is modeled after.

In some embodiments, each line in the graphical user interface may be initially set to a default state. For example, all the lines in the graphical user interface may initially be displayed as bold lines (e.g., set to a “strong” display state), or all the lines may initially be displayed so that they are indetectable against the background (e.g., set to a “none” display state). In some embodiments, each line in the graphical user interface may be individually set to a default state (e.g., a default state may be configured for each line). For example, one line in the graphical user interface may initially be displayed as a bold line (e.g., set to a strong display state) while the other lines may initially be displayed so that they are indetectable against the background (e.g., set to a “none” display state).

In some embodiments, the lines in the graphical user interface may be interactable elements. In some embodiments, the display states of the lines may be changeable based on user inputs received through the graphical user interface. For example, each line may be clickable, and clicking on a line may change its display state to a different state selected from among a set of possible display states. In some embodiments, clicking on a line may cycle its display state through some or all of the possible display states. In some embodiments, there may be separate user interface elements for changing the display states of the lines. For example, next to each line may be a button (e.g., to cycle its display state through some or all of the possible display states), or two buttons (e.g., to cycle its display state back and forth through some or all of the possible display states), or a drop-down menu (e.g., for selecting a display state from among some or all of the possible display states), and so forth.

In some embodiments, clicking on a line in the graphical user interface may select it. Selecting a line may change the display state of the line temporarily (e.g. the line could become highlighted) to indicate to the user that the line is selected. A user may be able to change the display state of the line once it becomes selected. For example, once a line is selected, clicking on the line again the line may cycle its display state to the next possible display state. As another example, once a line is selected, buttons or a scroll bar may appear next to the line, and the user may be able to use that to cycle forwards and backwards through the possible display states (e.g., a scroll bar could allow the user to scrub up and down, and scrubbing up may make the line darker while scrubbing down may make the line fainter).

In some embodiments, the graphical user interface may be configured to be displayed on a device with a touch-sensitive surface (e.g., a touchscreen) and one or more sensors for detecting the presence and intensities of inputs on the touch-sensitive surface. The graphical user interface may have interactive zones (e.g., touch-sensitive) that can be touched or pressed and correspond to the result zones of the diagnostic test, such as interactive zones over the lines that correspond to the lines of a lateral flow test. For example, a user touching or pressing on a line in the graphical user interface may change its display state to a different state selected from among a set of possible display states, such as by cycling its display state through some or all of the possible display states. In some embodiments, a user's touch or finger press may have a similar effect as a mouse click and/or hold, so it may be easy to adapt the graphical user interface for use with touchscreens (e.g., on a mobile device), for use with a website or web-application (e.g., on a browser), for use with a standalone application, and so forth.

In some embodiments, the graphical user interface may be further configured with additional user interface elements to better resemble the results window of a particular lateral flow test. For example, there may be a border enclosing or surrounding the lines on the graphical user interface (e.g., that resembles the casing surrounding the results window of the actual lateral flow test), there may be labels next to the lines on the graphical user interface (e.g., alphanumeric characters, shapes, colors, etc., to help identify and distinguish the lines from one another), and so forth.

In some embodiments, the graphical user interface may provide instructions or guidance to the user (e.g., “Please click the lines until they match what you see on your test”). Thus, the user can interact with the graphical user interface and change the display state of each line until all of the lines resemble the lines on an actual lateral flow test in their possession. In some embodiments, the graphical user interface may have a user interface element, such as a button (e.g., a “save” or “enter” or “continue” button), that a user can interact with once all the lines in the interface sufficiently match or resemble the display state of their lateral flow test. This can end the matching process and begin next steps. For example, the display states of the lines can be processed, converted, saved, and/or interpreted, and so forth.

In some embodiments, the display states of the lines in the graphical user interface may be processed or converted into a machine-readable format. For example, each line may have a name or label (e.g., “C”, “A”, “B”, etc.) and the display state of each line may be represented by a value (e.g., alphanumeric values such as “0”, “1”, “2”, etc. representing display states that differ by color intensity). Thus, the display states of all the lines (e.g., the entire state of the widget) can be encoded into a table or any other suitable data format (e.g., data objects consisting of attribute-value pairs, such as in JSON format). This may provide a very compact and efficient method of storing and communicating the data (e.g., reduced file size).

In some embodiments, the display states of the lines in the graphical user interface may be interpreted based on a set of logic. In some embodiments, a possible interpretation of the diagnostic test results window can be generated, and the possible interpretation may be suggested to the user on the graphical user interface (e.g., for the user to confirm or deny). In some embodiments, various prompts and recommendations (e.g., for next steps or further action) can be made to the user in the graphical user interface.

In some embodiments, once the matching process has ended (e.g., the user has indicated that the display states of all the lines in the graphical user interface resemble their diagnostic test results), a screen grab of the user interface may be captured on the user's device and sent to a supervising user (e.g., a proctor) for review. Alternatively, the display states of the lines in the graphical user interface may be processed or converted into a machine-readable format and sent to the supervising user's device, which may convert the machine-readable data back into lines displayed on a graphical user interface for the supervising user to review the display states.

In some embodiments, the user may be requested to capture a snapshot of the results window of their diagnostic test (e.g., using a camera on their device), which can be sent to a supervising user for review. This can be done before or after the matching process. For example, after the user has indicated that the display states of all the lines in the graphical user interface resemble their diagnostic test results, the user may be prompted to capture a snapshot of the results window of their diagnostic test using a camera on their device.

In some embodiments, a supervising user may be presented with a similar graphical user interface that may also be used to digitally recreate the display state of any suitable diagnostic test result (e.g., it may have non-overlapping lines that correspond to lines in the results window of the diagnostic test). The supervising user may be presented with the snapshot of the results window that was provided by the user, and the supervising user may be asked to recreate the results window in the graphical user interface (e.g., by interacting with the lines to change their display states) just as the user did. In some embodiments, the display states of the lines on the graphical user interface shown to the supervising user may be saved, processed, and/or converted (e.g., into a machine-readable format). The data from the supervising user may be compared to the data from the user (e.g., on a central server). In some embodiments, the results of the comparison may be used to progress the user onto a next step or to initiate a remediation process (e.g., have the user recreate the appearance of the results window in the user interface again).

In other alternative embodiments, a supervising user may instead be presented with a similar graphical user interface with lines that are pre-configured with the display states submitted by the user. For instance, the supervising user's device may be sent, in a machine-readable format from a central server, the display states of the lines entered by the user, which can then be used to set the display states of the lines in the user interface to those that were submitted by the user. The supervising user may be presented with the snapshot of the results window that was provided by the user, and the supervising user may be asked if the lines in the user interface match those in the snapshot (e.g., “Does this represent the pictured result?”). In some embodiments, the selection by the supervising user may be used to progress the user onto a next step or to initiate a remediation process (e.g., have the user recreate the appearance of the results window in the user interface again). For example, if the supervising user's selection and the user's selection do not match, then a prompt can be raised to the user (e.g., “Are you sure this is what you saw?”) and the user may be offered the ability to recreate the appearance of the results window again.

In some embodiments, certain logic and steps described herein may be performed on the user's device (and/or the supervising user's device), while in other embodiments, the logic and steps may be performed remotely (e.g., by a central server or in the cloud). For example, after the user recreates the test results window in the user interface and locks in the overall display states for submission, the user's device may interpret the submitted display states of the lines and generate a possible interpretation of the diagnostic test results (e.g., a positive result for a target analyte in the sample). Alternatively, the submitted display states for the lines may be transmitted in a machine-readable format to a central server or the cloud for interpretation, and a possible interpretation of the diagnostic test results may be sent back to the user's device. Transmitting the display states in a machine-readable format may be more efficient (e.g., reduced size compared to an image or screen grab of the user interface). However, the user device may still have to transmit an image or snapshot of the actual diagnostic test results (e.g., to a supervising user for comparison purposes, to the central server or the cloud to be forwarded to the supervising user, and so forth).

As another example, in some embodiments, in which the supervising user is tasked with using a graphical user interface to digitally recreate the display state of the actual diagnostic test result possessed by the user (e.g., captured by an image or snapshot), the supervising user's device may make a comparison between the display states of the lines submitted by the supervising user and the display states of the lines submitted by the user (e.g., the supervising user's device can be provided with the display states submitted by the user in a machine-readable format by a central server or the cloud). Or, the supervising user's device may transmit the display states for the lines submitted by the supervising user to the central server or the cloud for the comparison. There may be different advantages to performing certain logic and steps locally on a user device or remotely (e.g., on a central server, or in the cloud). For instance, performing steps locally may reduce the time spent by the user, whereas performing steps remotely may be more secure and/or provide technological benefits downstream (e.g., transmitting the display states for the lines submitted by the supervising user provides an additional datapoint on how the actual diagnostic test results possessed by the user may be visually interpreted by a human being, which may be useful for training machine learning models for future computer vision applications).

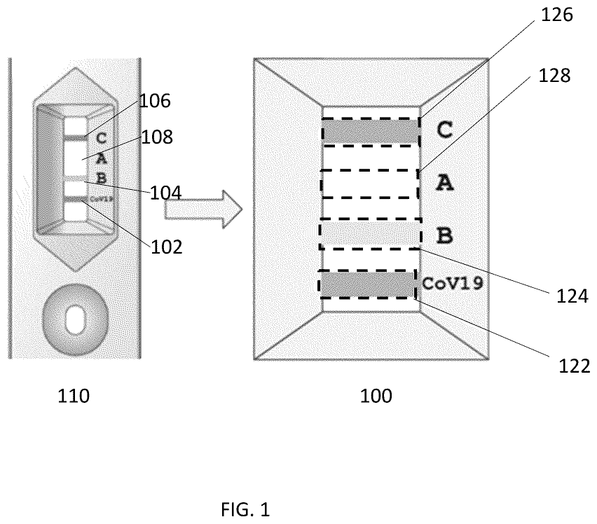

FIG. 1 depicts a non-limiting example of a results re-creation widget 100 mimicking the results of a real diagnostic test 110. The results re-creation widget 100 may be a graphical user interface or an interactable digital object within a graphical user interface which can be designed to resemble the results window of the real diagnostic test 110. In this non-limiting example, the real diagnostic test 110 is a combination COVID-19 and Flu antigen lateral flow test. The real diagnostic test 110 provides a graphical representation of test results, with the presence of a line being indicative of a possible positive result. A line in the control result zone 106 of the real diagnostic test 110 may indicate that the real diagnostic test 110 is valid. A line in the Flu-A result zone 108 of the real diagnostic test 110 may indicate the presence of a Flu-A antigen. A line in the Flu-B result zone 104 of the real diagnostic test 110 may indicate the presence of a Flu-B antigen. A line in the COVID-19 result zone 102 of the real diagnostic test 110 may indicate the presence of a COVID-19 antigen.

The results re-creation widget 100 may have interactive zones (e.g., touch sensitive) corresponding to each result zone of the real diagnostic test 110. For example, the re-creation widget 100 may have an interactive zone 126 corresponding to the control result zone 106, an interactive zone 128 corresponding to the Flu-A result zone 108, an interactive zone 124 corresponding to the Flu-B result zone 104, and an interactive zone 122 corresponding to the COVID-19 result zone 102. When triggered, each one of the interactive zones 126, 128, 124, and 122 may progress through multiple different display states to represent any arbitrary state of test results. In this non-limiting example, the interactive zones may display a strong-state represented by a bold line, a weak-state indicated by a faint line, and a none-state represented by no line.

In some embodiments, having three different display states (e.g., strong, weak, and none) may make it easier for a user without a lot of experience to recreate the results window of their diagnostic test, since there would only be three options that any line in the results window could be categorized under. In some embodiments, each line in the results window of a diagnostic test may require a simple binary interpretation (e.g., the line is either present or not). However, in such cases, it may still be useful for the graphical user interface to have more than two display states for the lines, since the extra information can be useful for analysis even though the additional display states may not change the interpretation of the results much (e.g., both strong and weak display states would still indicate that the line is present). For example, the extra information could be useful for improving the diagnostic tests or it could be useful for training machine learning models for future computer vision applications when combined with an image of the actual results window.

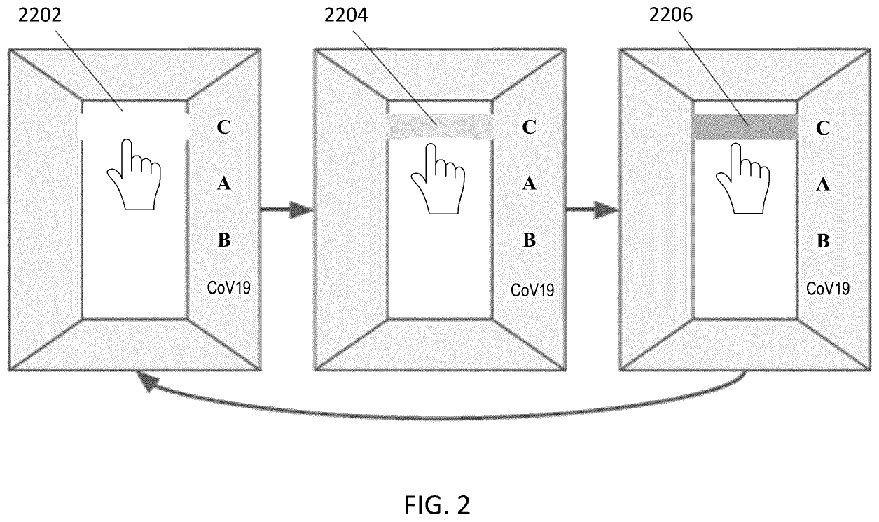

FIG. 2 is an example schematic diagram of a user inputting their real diagnostic test 110 results on the patient-facing side of the results re-creation widget 100. When the interactive zones 126, 128, 124, and 122 are triggered, they may progress to a different display state. In this non-limiting example, the display states progress cyclically upon each trigger event, starting with a none-state 2202 indicated by no line, followed by a weak-state 2204 indicated by a faint line, and then by a strong-state 2206 indicated by a bold line. By successively triggering each of the different interactive zones 126, 128, 124, and 122, a user of the results re-creation widget 100 can quickly mimic a real result from the real diagnostic test 110.

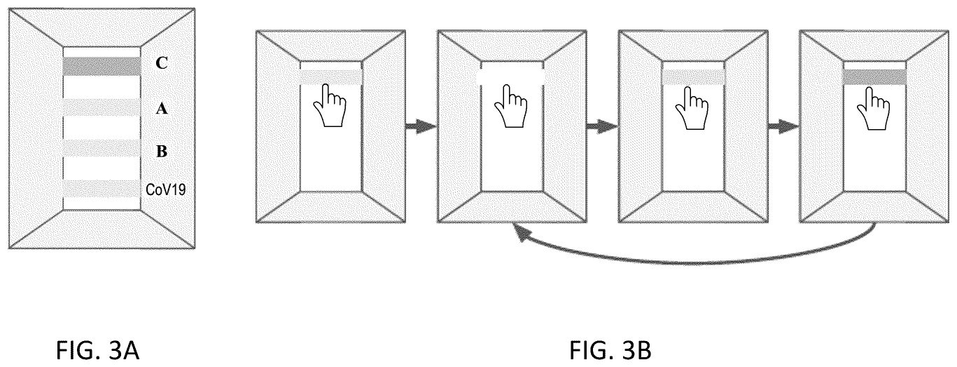

FIGS. 3A-B depict sample user interfaces of the patient-facing side of the results re-creation widget. The results re-creation widget's 100 user interface is an important consideration as the initial state that the interactive zones 126, 128, 124, and 122 are presented to a user may bias the results received. For example, if the start-screen of the results re-creation widget 100 initially presents to the user with all of the interactive zones 126, 128, 124, and 122, in the strong-state, the user would be penalized with having to trigger interactive zones 126, 128, 124, and 122 more times, which potentially biases towards users submitting false positive results. Additionally, if the start-screen of the results re-creation widget 100 initially presents to the user with all of the interactive zones 126, 128, 124, and 122, in the none-state, or with only the control interactive zone 128 in the strong state, the user could be confused as to which interactive zones 126, 128, 124, and 122 to trigger, which may result in false negative results. Similarly, if the user interface presents patients with a blank screen, (e.g., interactive zones 126, 128, 124, and 122 are not indicated), it would not be clear that the zones are interactable. FIG. 3A depicts one possible solution to this problem which is, at the start screen, to have the interactive zones 126, 128, 124, and 122 in a faint color that is different from the ones on the real diagnostic test 110, which shows the interactive zones may be triggered. In some embodiments, the interactive zones 126, 128, 124, and 122 may be flashing at the start screen, as depicted in FIG. 3B. Such slow flashing animation shows that lines are clickable and clarifies the user interface.

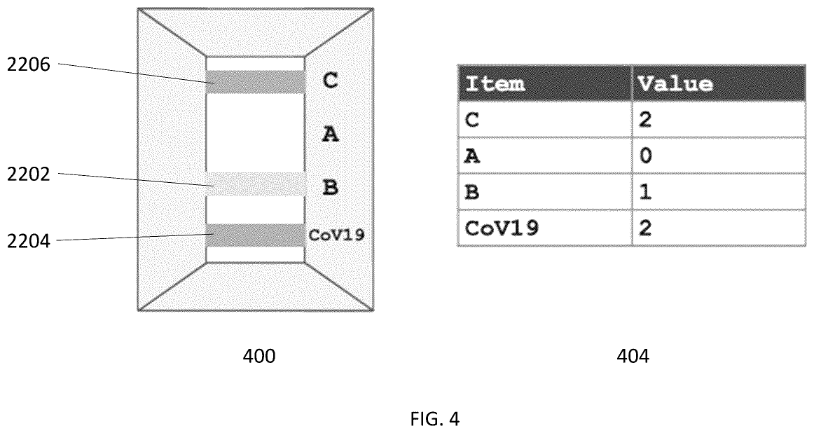

FIG. 4 illustrates how the display states of the graphical form 400 of the results re-creation widget 100 can be encoded into a machine-readable format, such as a tabular form 404. In this non-limiting example, the results re-creation widget 100 encodes a numerical value to each one of the display states. Thus, a numerical value can be associated with each line in the results window. The none-state 2022 is represented with a value of 0, the weak-state 2024 is associated with a value of 1, and the strong state 2206 is associated with a value of 2. Converting the graphical form 400 of the results into tabular form 404 in this manner advantageously provides a very compact method of storing and communicating the data.

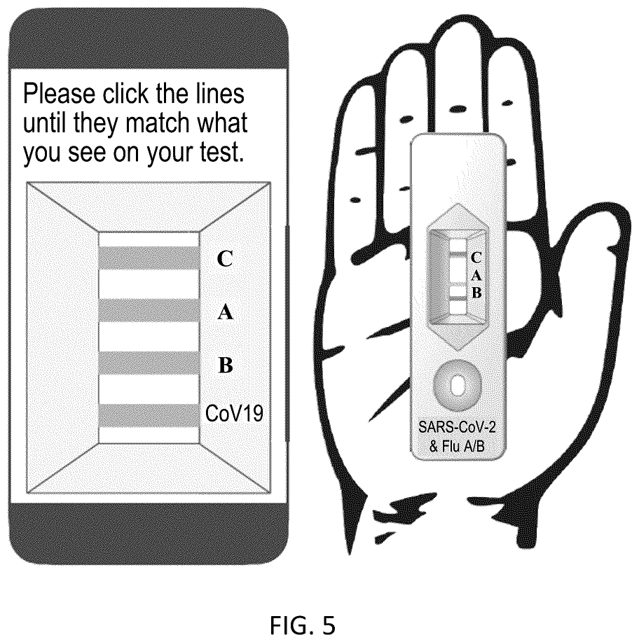

FIG. 5 depicts a sample perspective view of the patient-facing side of the results re-creation widget 100 in a mobile application 500. A user can be asked to use the widget to recreate their real diagnostic test 110 result as a means to communicate that result to a digital healthcare platform as part of a proctored or un-proctored experience. By using such a tool to submit their result, the user can bypass a potential large and complicated logic tree (e.g., the user asking themselves “How many lines do you see?”, followed by “Is there a line next to the letter “C” ?”, followed by, “Is there a line next to the letter “A” ?, followed by “Is there a line next to the letter “B” ?, followed by “Is there a line next to the symbol “CoV19” ?). In such an example, in addition to requiring many questions, it does not represent the difference between “strong” and “weak” lines without even further questions being asked.

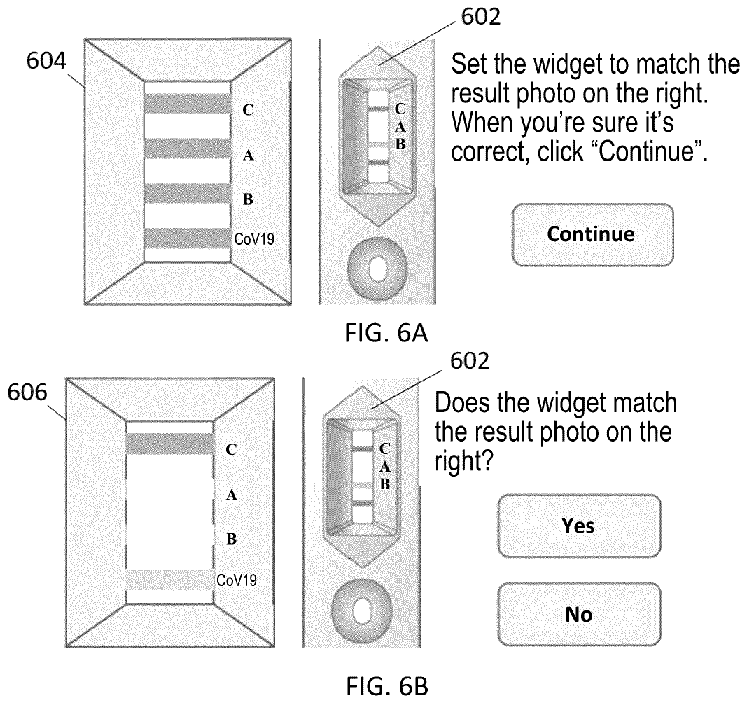

FIGS. 6A-6B depict sample user interfaces of the proctor-facing side having the results re-creation widget 100 to recreate the display states of the results of a real diagnostic test 110. FIG. 6A shows an example where the results re-creation widget 604 prompts a supervising user (e.g., a proctor or doctor) to input a test result based off an image 602 of a results window of a diagnostic test provided by a user (e.g., the real diagnostic test 110 result). First, the results re-creation widget 604 asks the proctor to recreate the pictured result 602 with the widget. Then the submitted display states in the re-creation widget 604 can be compared to the user's submitted result in the background, and either automatically advances if the user-submitted image 602 and supervising user's input are consistent, or starts result remediation if the supervising user's input and the user-submitted image 602 are not consistent.

FIG. 6B shows an example of a results re-creation widget 100 in which a supervising user is presented with a graphical representation 606 of results matching the user's submitted display states and is asked “does this represent the pictured result?” For instance, the supervising user's device may be sent the display states of the lines that were submitted by the user, which can be used to pre-configure the display states of the lines in the results re-creation widget 100 presented to the supervising user. The supervising user may be presented with multiple options to respond to the prompt, e.g., if “yes”, advance; if “no,” then the supervising user may be able to input their perceived result and start result remediation.

The processes described in relation to FIGS. 6A-6B above advantageously reduce the complexity of the test experience. They reduce the time required by the user and the proctor, reduce the proctor's cognitive load and capture an easily encoded result that can be used for training machine learning models for future computer vision applications.

### Computer Systems

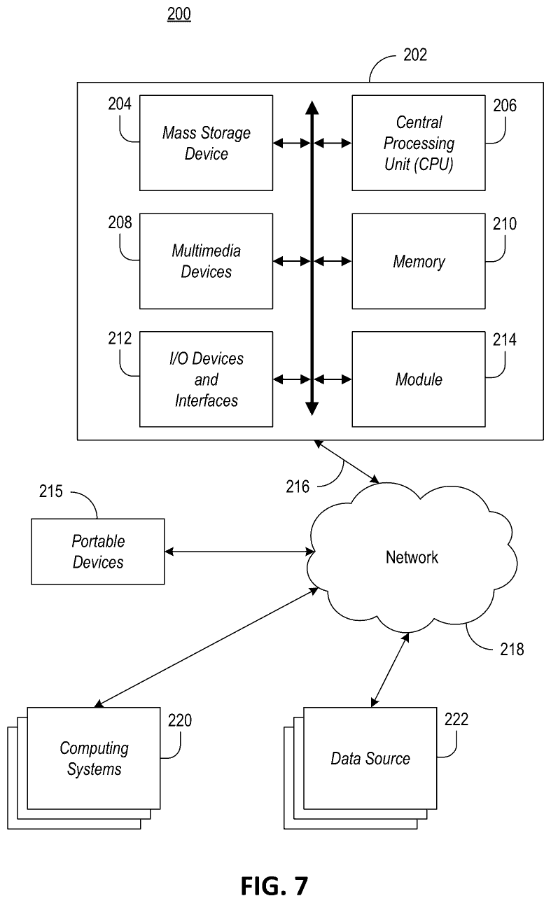

FIG. 7 is a block diagram depicting an embodiment of a computer hardware system configured to run software for implementing one or more embodiments of the health testing and diagnostic systems, methods, and devices disclosed herein. The example computer system 202 is in communication with one or more computing systems 220 and/or one or more data sources 222 via one or more networks 218. While FIG. 7 illustrates an embodiment of a computing system 202, it is recognized that the functionality provided for in the components and modules of computer system 202 may be combined into fewer components and modules, or further separated into additional components and modules.

The computer system 202 can comprise a module 214 that carries out the functions, methods, acts, and/or processes described herein. The module 214 is executed on the computer system 202 by a central processing unit 206 discussed further below.

In general, the word “module,” as used herein, refers to logic embodied in hardware or firmware or to a collection of software instructions, having entry and exit points. Modules are written in a program language, such as JAVA, C or C++, PYPHON or the like. Software modules may be compiled or linked into an executable program, installed in a dynamic link library, or may be written in an interpreted language such as BASIC, PERL, LUA, or Python. Software modules may be called from other modules or from themselves, and/or may be invoked in response to detected events or interruptions. Modules implemented in hardware include connected logic units such as gates and flip-flops, and/or may include programmable units, such as programmable gate arrays or processors.

Generally, the modules described herein refer to logical modules that may be combined with other modules or divided into sub-modules despite their physical organization or storage. The modules are executed by one or more computing systems and may be stored on or within any suitable computer readable medium or implemented in-whole or in-part within special designed hardware or firmware. Not all calculations, analysis, and/or optimization require the use of computer systems, though any of the above-described methods, calculations, processes, or analyses may be facilitated through the use of computers. Further, in some embodiments, process blocks described herein may be altered, rearranged, combined, and/or omitted.

The computer system 202 includes one or more processing units (CPU) 206, which may comprise a microprocessor. The computer system 202 further includes a physical memory 210, such as random-access memory (RAM) for temporary storage of information, a read only memory (ROM) for permanent storage of information, and a mass storage device 204, such as a backing store, hard drive, rotating magnetic disks, solid state disks (SSD), flash memory, phase-change memory (PCM), 3D XPoint memory, diskette, or optical media storage device. Alternatively, the mass storage device may be implemented in an array of servers. Typically, the components of the computer system 202 are connected to the computer using a standards-based bus system. The bus system can be implemented using various protocols, such as Peripheral Component Interconnect (PCI), Micro Channel, SCSI, Industrial Standard Architecture (ISA) and Extended ISA (EISA) architectures.

The computer system 202 includes one or more input/output (I/O) devices and interfaces 212, such as a keyboard, mouse, touch pad, and printer. The I/O devices and interfaces 212 can include one or more display devices, such as a monitor, which allows the visual presentation of data to a user. More particularly, a display device provides for the presentation of GUIs as application software data, and multi-media presentations, for example. The I/O devices and interfaces 212 can also provide a communications interface to various external devices. The computer system 202 may comprise one or more multi-media devices 208, such as speakers, video cards, graphics accelerators, and microphones, for example.

The computer system 202 may run on a variety of computing devices, such as a server, a Windows server, a Structure Query Language server, a Unix Server, a personal computer, a laptop computer, and so forth. In other embodiments, the computer system 202 may run on a cluster computer system, a mainframe computer system and/or other computing system suitable for controlling and/or communicating with large databases, performing high volume transaction processing, and generating reports from large databases. The computing system 202 is generally controlled and coordinated by an operating system software, such as z/OS, Windows, Linux, UNIX, BSD, SunOS, Solaris, MacOS, or other compatible operating systems, including proprietary operating systems. Operating systems control and schedule computer processes for execution, perform memory management, provide file system, networking, and I/O services, and provide a user interface, such as a graphical user interface (GUI), among other things.

The computer system 202 illustrated in FIG. 7 is coupled to a network 218, such as a LAN, WAN, or the Internet via a communication link 216 (wired, wireless, or a combination thereof). Network 218 communicates with various computing devices and/or other electronic devices. Network 218 is communicating with one or more computing systems 220 and one or more data sources 222. The module 214 may access or may be accessed by computing systems 220 and/or data sources 222 through a web-enabled user access point. Connections may be a direct physical connection, a virtual connection, and other connection type. The web-enabled user access point may comprise a browser module that uses text, graphics, audio, video, and other media to present data and to allow interaction with data via the network 218.

Access to the module 214 of the computer system 202 by computing systems 220 and/or by data sources 222 may be through a web-enabled user access point such as the computing systems' 220 or data source's 222 personal computer, cellular phone, smartphone, laptop, tablet computer, e-reader device, audio player, or another device capable of connecting to the network 218. Such a device may have a browser module that is implemented as a module that uses text, graphics, audio, video, and other media to present data and to allow interaction with data via the network 218.

The output module may be implemented as a combination of an all-points addressable display such as a cathode ray tube (CRT), a liquid crystal display (LCD), a plasma display, or other types and/or combinations of displays. The output module may be implemented to communicate with input devices 212 and they also include software with the appropriate interfaces which allow a user to access data through the use of stylized screen elements, such as menus, windows, dialogue boxes, tool bars, and controls (for example, radio buttons, check boxes, sliding scales, and so forth). Furthermore, the output module may communicate with a set of input and output devices to receive signals from the user.

The input device(s) may comprise a keyboard, roller ball, pen and stylus, mouse, trackball, voice recognition system, or pre-designated switches or buttons. The output device(s) may comprise a speaker, a display screen, a printer, or a voice synthesizer. In addition, a touch screen may act as a hybrid input/output device. In another embodiment, a user may interact with the system more directly such as through a system terminal connected to the score generator without communications over the Internet, a WAN, or LAN, or similar network.

In some embodiments, the system 202 may comprise a physical or logical connection established between a remote microprocessor and a mainframe host computer for the express purpose of uploading, downloading, or viewing interactive data and databases on-line in real time. The remote microprocessor may be operated by an entity operating the computer system 202, including the client server systems or the main server system, an/or may be operated by one or more of the data sources 222 and/or one or more of the computing systems 220. In some embodiments, terminal emulation software may be used on the microprocessor for participating in the micro-mainframe link.

In some embodiments, computing systems 220 who are internal to an entity operating the computer system 202 may access the module 214 internally as an application or process run by the CPU 206.

In some embodiments, one or more features of the systems, methods, and devices described herein can utilize a URL and/or cookies, for example for storing and/or transmitting data or user information. A Uniform Resource Locator (URL) can include a web address and/or a reference to a web resource that is stored on a database and/or a server. The URL can specify the location of the resource on a computer and/or a computer network. The URL can include a mechanism to retrieve the network resource. The source of the network resource can receive a URL, identify the location of the web resource, and transmit the web resource back to the requestor. A URL can be converted to an IP address, and a Domain Name System (DNS) can look up the URL and its corresponding IP address. URLs can be references to web pages, file transfers, emails, database accesses, and other applications. The URLs can include a sequence of characters that identify a path, domain name, a file extension, a host name, a query, a fragment, scheme, a protocol identifier, a port number, a username, a password, a flag, an object, a resource name and/or the like. The systems disclosed herein can generate, receive, transmit, apply, parse, serialize, render, and/or perform an action on a URL.

A cookie, also referred to as an HTTP cookie, a web cookie, an internet cookie, and a browser cookie, can include data sent from a website and/or stored on a user's computer. This data can be stored by a user's web browser while the user is browsing. The cookies can include useful information for websites to remember prior browsing information, such as a shopping cart on an online store, clicking of buttons, login information, and/or records of web pages or network resources visited in the past. Cookies can also include information that the user enters, such as names, addresses, passwords, credit card information, etc. Cookies can also perform computer functions. For example, authentication cookies can be used by applications (for example, a web browser) to identify whether the user is already logged in (for example, to a web site). The cookie data can be encrypted to provide security for the consumer. Tracking cookies can be used to compile historical browsing histories of individuals. Systems disclosed herein can generate and use cookies to access data of an individual. Systems can also generate and use JSON web tokens to store authenticity information, HTTP authentication as authentication protocols, IP addresses to track session or identity information, URLs, and the like.

The computing system 202 may include one or more internal and/or external data sources (for example, data sources 222). In some embodiments, one or more of the data repositories and the data sources described above may be implemented using a relational database, such as DB2, Sybase, Oracle, CodeBase, and Microsoft® SQL Server as well as other types of databases such as a flat-file database, an entity relationship database, and object-oriented database, and/or a record-based database.

The computer system 202 may also access one or more databases 222. The databases 222 may be stored in a database or data repository. The computer system 202 may access the one or more databases 222 through a network 218 or may directly access the database or data repository through I/O devices and interfaces 212. The data repository storing the one or more databases 222 may reside within the computer system 202.

### ADDITIONAL EMBODIMENTS

In the foregoing specification, the systems and processes have been described with reference to specific embodiments thereof. It will, however, be evident that various modifications and changes may be made thereto without departing from the broader spirit and scope of the embodiments disclosed herein. The specification and drawings are, accordingly, to be regarded in an illustrative rather than restrictive sense.

Indeed, although the systems and processes have been disclosed in the context of certain embodiments and examples, it will be understood by those skilled in the art that the various embodiments of the systems and processes extend beyond the specifically disclosed embodiments to other alternative embodiments and/or uses of the systems and processes and obvious modifications and equivalents thereof. In addition, while several variations of the embodiments of the systems and processes have been shown and described in detail, other modifications, which are within the scope of this disclosure, will be readily apparent to those of skill in the art based upon this disclosure. It is also contemplated that various combinations or sub-combinations of the specific features and aspects of the embodiments may be made and still fall within the scope of the disclosure. It should be understood that various features and aspects of the disclosed embodiments can be combined with, or substituted for, one another in order to form varying modes of the embodiments of the disclosed systems and processes. Any methods disclosed herein need not be performed in the order recited. Thus, it is intended that the scope of the systems and processes herein disclosed should not be limited by the particular embodiments described above.

It will be appreciated that the systems and methods of the disclosure each have several innovative aspects, no single one of which is solely responsible or required for the desirable attributes disclosed herein. The various features and processes described above may be used independently of one another or may be combined in various ways. All possible combinations and sub-combinations are intended to fall within the scope of this disclosure.

Certain features that are described in this specification in the context of separate embodiments also may be implemented in combination in a single embodiment. Conversely, various features that are described in the context of a single embodiment also may be implemented in multiple embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination. No single feature or group of features is necessary or indispensable to each and every embodiment.

It will also be appreciated that conditional language used herein, such as, among others, “can,” “could,” “might,” “may,” “for example,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. In addition, the term “or” is used in its inclusive sense (and not in its exclusive sense) so that when used, for example, to connect a list of elements, the term “or” means one, some, or all of the elements in the list. In addition, the articles “a,” “an,” and “the” as used in this application and the appended claims are to be construed to mean “one or more” or “at least one” unless specified otherwise. Similarly, while operations may be depicted in the drawings in a particular order, it is to be recognized that such operations need not be performed in the particular order shown or in sequential order, or that all illustrated operations be performed, to achieve desirable results. Further, the drawings may schematically depict one or more example processes in the form of a flowchart. However, other operations that are not depicted may be incorporated in the example methods and processes that are schematically illustrated. For example, one or more additional operations may be performed before, after, simultaneously, or between any of the illustrated operations. Additionally, the operations may be rearranged or reordered in other embodiments. In certain circumstances, multitasking and parallel processing may be advantageous. Moreover, the separation of various system components in the embodiments described above should not be understood as requiring such separation in all embodiments, and it should be understood that the described program components and systems may generally be integrated together in a single software product or packaged into multiple software products. Additionally, other embodiments are within the scope of the following claims. In some cases, the actions recited in the claims may be performed in a different order and still achieve desirable results.

Further, while the methods and devices described herein may be susceptible to various modifications and alternative forms, specific examples thereof have been shown in the drawings and are herein described in detail. It should be understood, however, that the embodiments are not to be limited to the particular forms or methods disclosed, but, to the contrary, the embodiments are to cover all modifications, equivalents, and alternatives falling within the spirit and scope of the various implementations described and the appended claims. Further, the disclosure herein of any particular feature, aspect, method, property, characteristic, quality, attribute, element, or the like in connection with an implementation or embodiment can be used in all other implementations or embodiments set forth herein. Any methods disclosed herein need not be performed in the order recited. The methods disclosed herein may include certain actions taken by a practitioner; however, the methods can also include any third-party instruction of those actions, either expressly or by implication. The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers and should be interpreted based on the circumstances (for example, as accurate as reasonably possible under the circumstances, for example ±5%, ±10%, ±15%, etc.). For example, “about 3.5 mm” includes “3.5 mm.” Phrases preceded by a term such as “substantially” include the recited phrase and should be interpreted based on the circumstances (for example, as much as reasonably possible under the circumstances). For example, “substantially constant” includes “constant.” Unless stated otherwise, all measurements are at standard conditions including temperature and pressure.

As used herein, a phrase referring to “at least one of” a list of items refers to any combination of those items, including single members. As an example, “at least one of: A, B, or C” is intended to cover: A, B, C, A and B, A and C, B and C, and A, B, and C. Conjunctive language such as the phrase “at least one of X, Y and Z,” unless specifically stated otherwise, is otherwise understood with the context as used in general to convey that an item, term, etc. may be at least one of X, Y or Z. Thus, such conjunctive language is not generally intended to imply that certain embodiments require at least one of X, at least one of Y, and at least one of Z to each be present. The headings provided herein, if any, are for convenience only and do not necessarily affect the scope or meaning of the devices and methods disclosed herein.

Accordingly, the claims are not intended to be limited to the embodiments shown herein but are to be accorded the widest scope consistent with this disclosure, the principles and the novel features disclosed herein.

We claim:

1. A computer-implemented method for digitally recreating a display state of a test result in a graphical user interface, the method comprising:
displaying a graphical user interface on a screen, wherein the graphical user interface comprises a plurality of non-overlapping bands that each have a display state selected from among a set of possible display states, and wherein the display state of each band of the plurality of bands is initially set to a default state selected from among the set of possible display states;
detecting a first user input associated with a first band of the plurality of bands;
upon detecting the first user input, updating the displayed graphical user interface by changing the display state of the first band to a different state selected from among the set of possible display states;
detecting a second user input indicating that the plurality of bands in the graphical user interface digitally recreates the display state of the test result; and
upon detecting the second user input, converting the display states of the plurality of bands into a machine-readable format.

2. The computer-implemented method of claim 1, further comprising generating a possible interpretation of the test result based on the converted display states of the plurality of bands.

3. The computer-implemented method of claim 1, wherein each band of the plurality of bands comprises an interactable region, and wherein the first user input comprises an interaction with the interactable region of the first band.

4. The computer-implemented method of claim 1, wherein the test result is a diagnostic test result of a lateral flow test.

5. The computer-implemented method of claim 1, wherein the graphical user interface further comprises:
a plurality of labels corresponding to the plurality of bands, wherein each label uniquely identifies a corresponding band of the plurality of bands; and
a border enclosing the plurality of bands.

6. The computer-implemented method of claim 1, wherein the screen is a touchscreen, and wherein the first user input involves touching the first band on the screen.

7. The computer-implemented method of claim 1, wherein each display state in the set of possible display states differs on color intensity.

8. The computer-implemented method of claim 1, wherein the machine-readable format for the converted display states of the plurality of bands comprises an alphanumeric value for each band of the plurality of bands, wherein the alphanumeric value corresponds to the display state of that band.

9. The computer-implemented method of claim 1, further comprising:
updating the graphical user interface to request an image of the test result;
capturing the image of the test result via a camera on a user device; and
transmitting, to a server, the image of the test result and the converted display states.

10. The computer-implemented method of claim 1, further comprising:
updating the graphical user interface to request an image of the test result;
capturing the image of the test result via a camera on a user device; and
transmitting, to a supervising user device, the image of the test result and the converted display states.

11. A computer readable, non-transitory storage medium having a computer program stored thereon for causing a suitably programmed computer system to process by one or more processors computer-program code to perform a method for digitally recreating a display state of a test result in a graphical user interface, the method comprising:
displaying a graphical user interface on a screen, wherein the graphical user interface comprises a plurality of non-overlapping bands that each have a display state selected from among a set of possible display states, and wherein the display state of each band of the plurality of bands is initially set to a default state selected from among the set of possible display states;
detecting a first user input associated with a first band of the plurality of bands;
upon detecting the first user input, updating the displayed graphical user interface by changing the display state of the first band to a different state selected from among the set of possible display states;
detecting a second user input indicating that the plurality of bands in the graphical user interface digitally recreates the display state of the test result; and
upon detecting the second user input, converting the display states of the plurality of bands into a machine-readable format.

12. The computer readable, non-transitory storage medium of claim 11, wherein the method further comprises generating a possible interpretation of the test result based on the converted display states of the plurality of bands.

13. The computer readable, non-transitory storage medium of claim 11, wherein the screen is a touchscreen, and wherein the first user input involves touching the first band on the screen.

14. The computer readable, non-transitory storage medium of claim 11, wherein each display state in the set of possible display states differs on color intensity.

15. A computer-implemented method for digitally recreating a display state of a test result in a graphical user interface, the method comprising:
generating data for displaying a graphical user interface on a screen of a user device, wherein the graphical user interface comprises a plurality of non-overlapping bands that each have a display state selected from among a set of possible display states, and wherein the display state of each band of the plurality of bands is initially set to a default state selected from among the set of possible display states;
transmitting the graphical user interface data to the user device;
receiving, from the user device, a first user input associated with a first band of the plurality of bands;
upon receiving the first user input, transmitting first update data to the user device for updating the displayed graphical user interface by changing the display state of the first band to a different state selected from among the set of possible display states; and
receiving, from the user device, the display states of the plurality of bands converted into a machine-readable format.

16. The computer-implemented method of claim 15, further comprising:
generating a possible interpretation of the test result based on the received display states of the plurality of bands;
transmitting second update data to the user device for updating the display graphical user interface to indicate the possible interpretation of the test result.

17. The computer-implemented method of claim 15, further comprising:
receiving, from user device, an image of the test result captured via a camera on the user device; and
transmitting, to a supervising user device, the image of the test result and the converted display states of the plurality of bands.

18. The computer-implemented method of claim 15, wherein the machine-readable format for the converted display states of the plurality of bands comprises an alphanumeric value for each band of the plurality of bands, wherein the alphanumeric value corresponds to the display state of that band.

19. The computer-implemented method of claim 15, wherein each display state in the set of possible display states differs on color intensity.

20. The computer-implemented method of claim 15, wherein the test result is a diagnostic test result of a lateral flow test.
